(19) United States
(12) Patent Application Publication
(10) Pub. No.: US 2024/0252246 A1
(43) Pub. Date: Aug. 1, 2024

(54) IMPLANT MANUFACTURING METHOD FOR DESIGNING IMPLANT ON BASIS OF MEDICAL IMAGE DATA AND VIRTUAL IMAGE DATA

(71) Applicant: ANYMEDI INC., Seoul (KR)

(72) Inventors: Gukbae KIM, Seoul (KR); Seunghyun CHOI, Seoul (KR); Hye Sung LEE, Seoul (KR); Bong Saeng KIM, Namyangju-si, Gyeonggi-do (KR)

(21) Appl. No.: 18/038,624

(22) PCT Filed: Nov. 24, 2021
(86) PCT No.: PCT/KR2021/017352

§ 371 Date: Feb. 12, 2024

(30) Foreign Application Priority Data
Nov. 24, 2020 (KR) 10-2020-0158811

(51) Int. Cl. A61B 34/10; A61F 2/18; B33Y 50/00; G06F 30/12; G16H 50/50

(52) U.S. Cl. CPC A61B 34/10; A61F 2/186; G06F 30/12; G16H 50/50; A61B 2034/104; A61B 2034/105; A61B 2034/108; A61F 2240/002; B33Y 50/00

(57) ABSTRACT

The present invention relates to a method for fabricating an implant, comprising: (a) acquiring patient medical imaging data to define the appearance prior to the implant's insertion and a seating surface where the implant is seated, (b) acquiring a virtual image data defining the appearance after the implant's insertion, and (c) designing the implant based on the acquired patient medical imaging data and the virtual imaging data, wherein, the step of (c) includes determining a shape and height of the implant based on a difference between an appearance prior to the implant's insertion and an appearance after the implant's insertion, based on a seating surface defined from the acquired patient medical imaging data. This allows for a more comfortable fit of the implant to the nasal bone and cartilage, while achieving a post-operative appearance that is consistent with the intentions of the surgeon and patient.

### FIELD OF THE INVENTION

The present invention relates to a method of fabricating an implant, and more particularly to a method of fabricating an implant, wherein the implant is designed based on medical imaging data and virtual imaging data.

### DISCUSSION OF THE RELATED ART

In recent years, there has been an explosion of interest in cosmetic surgery, along with an increased focus on beauty. One of the most popular facial procedures is rhinoplasty, which involves inserting an artificial filler such as silicone implant inside the nose to heighten the external appearance of the nose.

Traditionally, rhinoplasty surgery has been performed by selecting an implant that matches the shape of the patient's nose from a set of three prostheses of varying lengths and thicknesses, checking the bending of the nasal bones and cartilage during the surgery, and sculpting an internal shape in which the implant is placed.

However, the problem with this method is that it is time-consuming to carve, and it is not easy to create the desired shape of the implant with manual carving. In addition, if the implant does not fit closely to the nasal bone and cartilage and creates a space between them, side effects such as infection and location change can occur.

As a result, there has been a need for a method of fabricating implants that includes a method of designing implants that is more comfortable and allows the implant to adhere to the nasal bone and cartilage while achieving a post-operative appearance that meets the intent of the surgeon and patient.

### SUMMARY OF THE INVENTION

### Technical Solution

Accordingly, it is an object of the present disclosure to provide a method for fabricating an implant, comprising: (a) acquiring patient medical imaging data to define the appearance prior to the implant's insertion and a seating surface where the implant is seated, (b) acquiring a virtual image data defining the appearance after the implant's insertion, and (c) designing the implant based on the acquired patient medical imaging data and the virtual imaging data, wherein, the step of (c) includes determining a shape and height of the implant based on a difference between an appearance prior to the implant's insertion and an appearance after the implant's insertion, based on a seating surface defined from the acquired patient medical imaging data.

In addition, a method for fabricating an implant may further include in the step of (c), (i) with respect to at least one point (A) of the appearance prior to the implant's insertion, comprises: determining at least one point (B) of the appearance after the implant's insertion that defines a vector from the appearance prior to the implant's insertion to the appearance after the implant's insertion, determining at least one point (C) of a seating surface where the implant is seated that corresponds to the point (A) of the appearance prior to the implant's insertion, and determining at least one point (D), in space as an element constituting the implant, from the determined point (C) on the seating surface where the implant is seated based on the vector from the appearance prior to the implant's insertion to the appearance after the implant's insertion, and (ii) repeating the step of (i) for a plurality of other points of the appearance prior to the implant's insertion.

In addition, at least one point (B) of the appearance after the implant's insertion may be determined based on the shortest distance from the point (A) of the appearance prior to the implant's insertion to the appearance after the implant's insertion.

In another aspect of the present disclosure, a method for fabricating an implant may include the point (B) of the appearance after the implant's insertion corresponds to a foot of perpendicular line made to the appearance after the implant's insertion from the point (A) of the appearance prior to the implant's insertion, or the point (A) of the appearance prior to the implant's insertion corresponds to a foot of perpendicular line made to the appearance prior to the implant's insertion from the point (B) of the appearance after the implant's insertion.

In another aspect of the present disclosure, a method for fabricating an implant may include the point (C) on the seating surface where the implant is seated corresponds to a foot of perpendicular line made to the appearance prior to the implant's insertion from the point (A) of the appearance prior to the implant's insertion.

To achieve these objects and other advantages and in accordance with the purpose of the invention, a method for fabricating an implant, the step of determining at least one point (D), in space as an element constituting the implant, from the determined point (C) on the seating surface where the implant is seated, is characterized by parallel shifting the vector which is from the appearance prior to the implant's insertion to the appearance after the implant's insertion, to a vector with a starting point being the point (C) on the seating surface where the implant is seated.

In another aspect of the present disclosure, a method for fabricating an implant may include the steps of (i) and (ii), characterized in that the steps of (i) and (ii) performs to each cross-section perpendicular to the longitudinal direction of the implant.

In addition, a method for fabricating an implant may include adjusting the shape of the implant by deleting the at least one point (D) determined in steps (i) and (ii) to be an element constituting the implant.

In another aspect of the present disclosure, a method for fabricating an implant may include a computer-readable recording medium recording a program which may perform the method above.

In another aspect of the present disclosure, a method for fabricating an implant may include a computer program stored on a medium for executing the method above in combination with hardware.

The technical subjects pursued in embodiments of the disclosure may not be limited to the above mentioned technical subjects, and other technical subjects which are not mentioned may be clearly understood, through the following descriptions, by those skilled in the art to which the disclosure pertains.

### BRIEF DESCRIPTION OF THE DRAWINGS

FIG. 1 is a flow diagram of a method of fabricating an implant according to the present invention.

FIG. 2 is a diagram illustrating one example of determining the height of an implant according to the present invention.

FIG. 3 is a diagram collectively illustrating the heights of implants determined in accordance with the present invention.

FIG. 4 is a drawing illustrating one form of an implant determined in accordance with the present invention from various perspectives.

FIG. 5 is a drawing showing an example of an implant manufactured in accordance with the present invention from various perspectives.

### DETAILED DESCRIPTION OF THE INVENTION

Hereinafter, exemplary embodiments of the present disclosure will be described in detail with reference to the accompanying drawings. However, the invention is not limited or defined by the exemplary embodiments. Identical reference numerals in each drawing designate members that perform substantially the same function.

The terminology used in this application is merely for the purpose of describing specific embodiments and is not intended to limit the invention. Unless explicitly indicating otherwise in the context, singular terms encompass plural forms.

The terms used in the invention were selected based on their widely used general meanings in present considering the functionality in the present invention, but they may vary depending on the intention of those skilled in the art, precedents, or the emergence of new technologies. Additionally, there may be instances where the applicant has arbitrarily chosen specific terms, and in such cases, the detailed meaning of those terms will be described in the relevant sections of the specification. Therefore, the terms used in the invention should be defined based not only on the literal meaning of the terms but also on the meanings they hold and the content throughout the invention.

When any part of the specification states that a certain component is “included” in another component, it means that, unless expressly stated otherwise, it does not exclude other components and may include additional components.

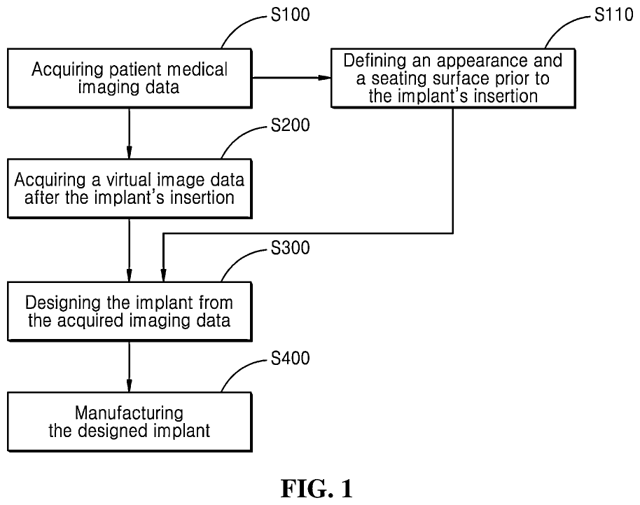

FIG. 1 is a flow diagram of a method of fabricating an implant according to the present invention.

Referring to the drawing, a method of manufacturing an implant of the present invention includes the steps of (a) acquiring patient medical imaging data (S100); (b) acquiring a virtual image data defining the appearance after the implant's insertion (S200); and (c) designing the implant based on the acquired patient medical imaging data and the virtual imaging data (S300). The method may further include, finally, the step of fabricating the designed implant (S400).

First, the step of (a) acquiring a patient medical image data (S100) is a step of acquiring patient medical image data that may define the appearance prior to the implant's insertion and the seating surface on which the implant is seated.

In this case, the patient's medical imaging data may be any imaging data, such as CT images, 3D scanning data, or the like, taken for the purpose of medical practice, that includes at least the surgical site, from which it is possible to determine the seating surface on which the implant is to be placed before surgery, and the three-dimensional overall contour of the surgical site before and/or after surgery.

The step S110 of defining the appearance before implant's insertion based on the patient medical imaging data includes converting it into a virtual 3D image, for example from a CT image, 3D scanning data, or the like. The conversion to a 3D image may be performed using a computing device equipped with software capable of performing such 3D image generation functions, such software may be implemented as conventional software, such as conventional CAD.

The step S110 of defining a seating surface for the implant based on the patient medical imaging data may also be performed using a computing device equipped with software capable of performing such a seating surface derivation function, such software may be implemented as conventional software, such as conventional CAD.

These 3D image generation functions and seating surface derivation functions may be separate pieces of software, or may be provided as a single, integrated piece of software. Further, the computing device on which such software(s) are mounted may be any computing device, such as a desktop computer, laptop computer, notebook, smartphone, or the like, or any device that may be integrated into a wired or wireless network.

Next, the step of (b) obtaining virtual image data defining the appearance after an implant's insertion (S200) may optionally include conversion to a virtual 3D image, for example, from a CT image, 3D scanning material, or the like. Additionally or separately, the conversion to a virtual 3D image may include the conversion to a virtual 3D image that reflects the intent of the patient and/or surgeon. The conversion to a 3D image may be performed using a computing device equipped with software capable of performing such 3D image generation functions, which may exist as software capable of performing functions to modify the 3D image corresponding to the appearance prior to the implant's insertion, separate from or in addition to the software that may be used to define the appearance prior to the implant's insertion. Such software may be implemented as conventional software, such as conventional CAD.

On the other hand, since the implant is not placed on the skin as it was before surgery, but rather under the skin, over bone, cartilage, and/or mucous membranes, the height of the implant cannot be determined simply based on the difference between the postoperative and preoperative appearance images. In order to minimize postoperative side effects, it is necessary to define a seating surface where the implant is to be seated comfortably as closely as possible to the real seating surface, and to determine the height of the implant based on the difference between the postoperative appearance and the preoperative appearance based on this defined seating surface.

The seating surface on which the implant is seated, can be derived for example, using the method described in Patent Application No. 2018-0056637, filed by the Applicant on May 17, 2018, entitled “NOSE IMPLANT MANUFACTURING METHOD”. In this method, the geometry of the nasal cartilage, which is not visible in the CT image, is modeled by applying anatomical elements from the images that are visible in the CT image—nasal bone images and nasal cavity images—to define a seating surface on which the implant is to be seated. Furthermore, it is disclosed to model the inner shape, which is the seating surface of the nose implant, based on the modeled nasal cartilage.

The step of (c) designing an implant (S300) based on the acquired patient medical image data and the hypothetical image data of the present invention includes determining a shape and height of the implant based on a difference between an appearance before insertion of the implant and an appearance after insertion of the implant based on a seating surface defined from the acquired patient medical image data. This design may also be performed using a computing device equipped with software capable of performing implant design functions, which may exist separately or as integrated software with the various software described above. Such software may be implemented as conventional software, such as conventional CAD.

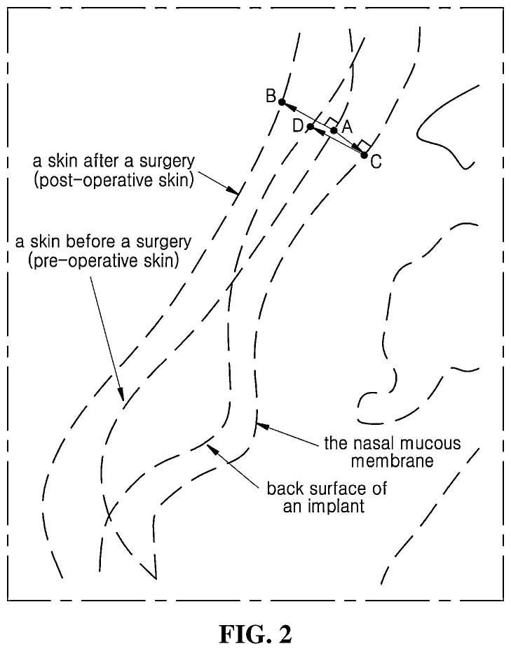

Determining the height of the implant based on the difference between the post-operative appearance image and the pre-operative appearance image with respect to the defined seating surface is illustrated, for example, in FIG. 2. Referring to FIG. 2, the dashed line labeled the nasal mucous membrane may be the seating surface on which the implant is to be placed, the dashed line labeled (hypothetical) a skin line after a surgery may be the post-operative appearance image, and the dashed line labeled a skin line before a surgery may be the pre-operative appearance image.

FIG. 2 illustrates acquiring the height of the implant from the difference between the skin before a surgery and the skin after a (hypothetical) surgery, for example, for any cross-section perpendicular to the direction of the length of the nose. Of course, the method for acquiring the difference may vary depending on how a point on the skin before a surgery is mapped to a point on the skin after a surgery, and how a point on the seating surface is mapped to these two points.

The steps for designing an implant according to the present invention may conceptually follow the following process.

(I) first, in a single cross-section perpendicular to the length of the nose, determining, for any one point of the appearance before insertion of the implant, any one point of the appearance after insertion of the implant, and any one point of the seating surface on which the implant is to be placed, and sequentially determining the height of the implant corresponding to any one point of the appearance before insertion of the implant, for all points that may be defined in the single cross-section.

(II) Secondly, repeat the above process for all perpendicular cross-sections in the lengthwise direction of the nose, which may be defined throughout the surgical area in which the implant may be inserted.

(III) Finally, the process of adjusting the shape of the implant can be optionally performed by deleting some of the height of the implants thus determined.

In this context, the height of the implant may be considered a descriptive term that focuses on the thickness of the implant relative to the seating surface, and the shape of the implant may be considered on the width of the implant when viewed in front of the patient.

In detail, for the process corresponding to (I),
(i) for any one point (A) of the appearance before the insertion of the implant: determining a point (B) of the appearance after the insertion of the implant that defines a vector from the appearance before the insertion of the implant to the appearance after the insertion of the implant; and determining any one point (C) on the seating surface on which the implant is seated, corresponding to the any one point (A) on which the appearance before the insertion of the implant; and determining any one point (D) in space from the any one point (C) of the seating surface on which the implant is seated, determined based on a vector from the appearance before the implant is inserted to the appearance after the implant is inserted, as a component of the implant. In this case, the term of vector is chosen to include direction, not merely distance.
(ii) repeating the step of (i) for a number of other points of the appearance prior to the insertion of the implant, in particular for one cross-section perpendicular to the length of the nose in which the corresponded any one point exists.

To illustrate this with reference to FIG. 2, a method of acquiring the height of an implant according to the present invention, first, locates a point B on the (hypothetical) post-operative skin that is closest from a point A on the pre-operative skin. The point A corresponds to the foot of the perpendicular line of the pre-operative skin of the vector from A to B (arrow AB).

In other words, one of the points (B) on an appearance after insertion of the implant may be determined based on the shortest distance from one of the points (A) on an appearance before insertion of the implant to an appearance after insertion of the implant. For this purpose, one of the points (B) of the appearance after the insertion of the implant may be set to correspond to the foot of the perpendicular line drawn from one of the points (A) on the appearance before the insertion of the implant to the appearance after the insertion of the implant, as shown in FIG., or alternatively, one of the points (A) of the appearance before the insertion of the implant may be set to correspond to the foot of the perpendicular line drawn from one of the points (B) of the appearance after the insertion of the implant to the appearance before the insertion of the implant.

Next, a foot of a perpendicular line (C) is drawn from the one of the points (A) on the pre-operative skin to the mucous membrane (arrow AC). In other words, the any one point (C) of) the seating surface on which the implant is seated may be set to correspond to a line of perpendicular drawn from any point (A) on the appearance before insertion of an implant to the nasal mucous membrane.

Finally, any one point that is moved by the vector corresponding to arrow AB from the any one point (C) on the nasal mucous membrane is defined as the height of the implant (D) (arrow CD). In other words, the vector from the appearance before the appearance of insertion of an implant to the appearance after the appearance of insertion of an implant may be moved parallel to the vector that starts from the any one point (C) on the seating surface which on the implant is seated.

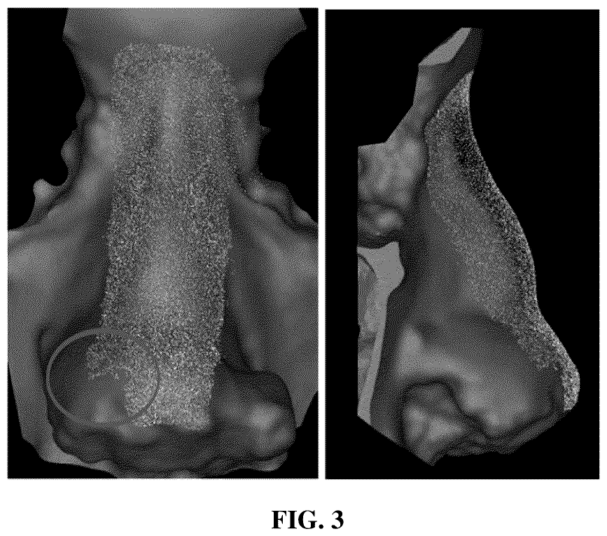

If the implant height (D) is thus determined for each of the perpendicular cross-sections of the nose lengthwise, for a plurality of points placed at appropriate predetermined intervals on the skin prior to surgery, the plurality of points for all or at least some of the multiple cross-sections, as shown in FIG. 3, constitutes any volume. Thus, this any volume may reflect to the designed (shape of) the implant.

On the other hand, the shape of the implant designed by the above process may not be smooth, as shown in the circled portion of FIG. 3. In such a case, if the implant is manufactured as designed according to the present invention, the rough edges may cause injury to the surgeon during the operation or leave scars on the patient's surgical site.

In such cases, the outline of the implant may optionally be adjusted by deleting the part of the outline of the any volume that has a point density below the predetermined standard, or by adjusting the width of the implant using the reference value set based on the accumulated data, or by free-curve modeling based on the skin shape after a surgery.

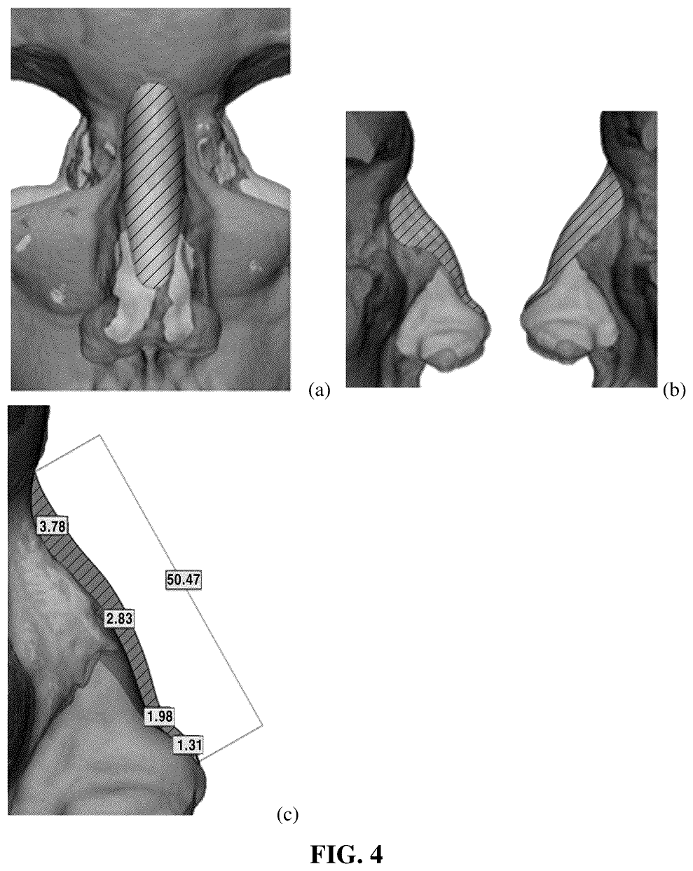

This completes the design of the implant (indicated by the diagonal lines), for example, as shown in FIG. 4, which illustrating the design of an implant according to the present invention.

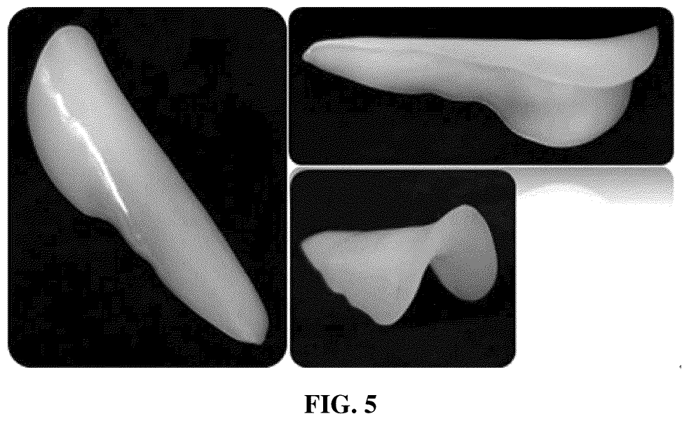

The method of fabricating an implant according to the present invention may further comprise the step of fabricating the designed implant according to the present invention (S400), wherein fabricating the designed implant may comprise directing the use of at least one of 3D printing, cutting, injection, mold, or vacuum forming, in conjunction with the implant design software described above. This completes the fabrication of the implant, as shown in FIG. 5.

Further, methods according to embodiments of the present invention may be implemented in the form of program instructions that may be executed through various computing devices and recorded on a computer-readable recording medium.

The program instruction forms, which may be collectively referred to as software, may include computer program, code, instructions, or one or more combinations thereof, which may configure a processing device to operate as desired, or may independently or collectively instruct a processing device. The software and/or data may be permanently or temporarily embodied in any type of machine, component, physical device, virtual equipment, computer storage medium, or apparatus for interpretation by, or for providing instructions or data to, a processing device. Software may also be distributed across networked computing devices and stored or executed in a distributed manner. The software and data may be stored on one or more computer-readable recording media.

The computer-readable recording medium may include program instructions, data files, data structures, and the like, singly or in combination. The program instructions recorded on the medium may be specifically designed and configured for the embodiment or may be known and available to those skilled in the art of computer software. Examples of computer-readable recording media include magnetic media such as hard disks, floppy disks, and magnetic tapes, optical media such as CD-ROMs and DVDs, magneto-optical media such as floptical disks, and hardware devices specifically configured to store and carry program instructions such as ROMs, RAMs, flash memory, and the like. Examples of program instructions include machine language code, such as that created by a compiler, as well as high-level language code that can be executed by a computer using an interpreter or the like. The hardware devices may be configured to operate as one or more software modules to perform the operations of the embodiments, and vice versa.

In summary, a method for designing an implant based on the medical imaging data and virtual imaging data of the present invention, and a method for fabricating the implant, enables the implant to adhere more comfortably to the nasal bones and cartilage while achieving a post-operative appearance that is consistent with the intentions of the operator and patient.

In general, terms used herein, particularly in the claims (e.g., in the body of the claims), are intended to be generally “open-ended” terms (e.g., “including” is to be construed as “including but not limited to,” “having” is to be construed as “having at least as much as,” and “comprising” is to be construed as “including but not limited to).” If a specific number is intended for an introduced claim term, such intent is expressly stated in the claim, and in the absence of such a statement, such intent is understood not to exist.

Only certain features of the present invention have been shown and described in this specification, and various modifications and changes may occur to those skilled in the art. It is therefore understood that the claims are intended to cover changes and modifications that fall within the spirit of the invention.

1. A method for fabricating an implant, comprising:
(a) acquiring patient medical imaging data to define the appearance prior to the implant's insertion and a seating surface where the implant is seated;
(b) acquiring a virtual image data defining the appearance after the implant's insertion; and
(c) designing the implant based on the acquired patient medical imaging data and the virtual imaging data;
wherein, the step of (c) includes determining a shape and height of the implant based on a difference between an appearance prior to the implant's insertion and an appearance after the implant's insertion, based on a seating surface defined from the acquired patient medical imaging data.

2. The method of claim 1, the step of (c) includes,
(i) with respect to at least one point (A) of the appearance prior to the implant's insertion, comprises:
determining at least one point (B) of the appearance after the implant's insertion that defines a vector from the appearance prior to the implant's insertion to the appearance after the implant's insertion;
determining at least one point (C) of a seating surface where the implant is seated that corresponds to the point (A) of the appearance prior to the implant's insertion; and
determining at least one point (D), in space as an element constituting the implant, from the determined point (C) on the seating surface where the implant is seated based on the vector from the appearance prior to the implant's insertion to the appearance after the implant's insertion;
and
(ii) repeating the step of (i) for a plurality of other points of the appearance prior to the implant's insertion.

3. The method of claim 2, characterized in that at least one point (B) of the appearance after the implant's insertion is determined based on the shortest distance from the point (A) of the appearance prior to the implant's insertion to the appearance after the implant's insertion.

4. The method of claim 2, the point (B) of the appearance after the implant's insertion corresponds to a foot of perpendicular line made to the appearance after the implant's insertion from the point (A) of the appearance prior to the implant's insertion, or
the point (A) of the appearance prior to the implant's insertion corresponds to a foot of perpendicular line made to the appearance prior to the implant's insertion from the point (B) of the appearance after the implant's insertion.

5. The method of claim 2, the point (C) on the seating surface where the implant is seated corresponds to a foot of perpendicular line made to the appearance prior to the implant's insertion from the point (A) of the appearance prior to the implant's insertion.

6. The method of claim 2, the step of determining at least one point (D), in space as an element constituting the implant, from the determined point (C) on the seating surface where the implant is seated, is characterized by parallel shifting the vector which is from the appearance prior to the implant's insertion to the appearance after the implant's insertion, to a vector with a starting point being the point (C) on the seating surface where the implant is seated.

7. The method of claim 6, the steps of (i) and (ii), characterized in that the steps of (i) and (ii) performs to each cross-section perpendicular to the longitudinal direction of the implant.

8. The method of claim 7, adjusting the shape of the implant by deleting the at least one point (D) determined in steps (i) and (ii) to be an element constituting the implant.

9. A non-transitory computer-readable recording medium recording a program performing the method of claim 1.

10. A computer program stored on a non-transitory medium for executing the method of claim 1 in combination with hardware.
